(19) United States
(12) Patent Application Publication
(10) Pub. No.: US 2023/0270500 A1
(43) Pub. Date: Aug. 31, 2023

(54) GENERATING AND DISPLAYING A RENDERING OF A LEFT ATRIAL APPENDAGE

(71) Applicant: KONINKLIJKE PHILIPS N.V., EINDHOVEN (NL)

(72) Inventors: Frank Michael Weber, Norderstedt (DE); Alasdair Iain Dow, Snohomish, WA (US); Eduardo Ortiz Vazquez, Cambridge, MA (US); Andrea Laghi, Melrose, MA (US); Arne Ewald, Hamburg (DE); Irina Waechter-Stehle, Hamburg (DE)

(21) Appl. No.: 18/012,294

(22) PCT Filed: Jun. 23, 2021
(86) PCT No.: PCT/EP2021/067098

§ 371 Date: Dec. 22, 2022

Related U.S. Application Data
(60) Provisional application No. 63/045,373, filed on Jun. 29, 2020.

(51) Int. Cl. A61B 34/10

(52) U.S. Cl. CPC A61B 34/10; G06T 2207/30048; A61B 2034/105

(57) ABSTRACT

A mechanism for generating a rendering of a left atrial appendage of a patient. The potential position of one or more interventional devices within the left atrial appendage is determined from model data that contains an anatomical model of the left atrial appendage. A rendering of the left atrial appendage is generated, from image data, and subsequently displayed. Visual parameters of the displayed rendering of the left atrial appendage are responsive to the determined potential position(s) for the one or more interventional devices.

### FIELD OF THE INVENTION

The present invention relates to the field of anatomical models, and in particular to renderings of anatomical structures.

### BACKGROUND OF THE INVENTION

There is an increasing trend in the performance of clinical procedures that involve the positioning of interventional devices within a left atrial appendage (LAA) of a patient, such as LAA occlusion. LAA occlusion helps prevent thrombi that lead to life-threating conditions, such as stroke. Proper understanding and sizing of the anatomy is crucial in order to select and position an appropriate LAA interventional device that makes the procedure successful.

Planning the position and size of a left atrial appendage interventional device is difficult, because the shape and size of the ostium (in which interventional device are normally positioned) vary between patients. Moreover, the LAA is not solely tube-like, but gradually gets wider towards the ostium. This requires a careful choice of the optimal position for an interventional device given the 3D shape of the ostium.

There is therefore a desire to provide a clinician or other user with data that facilitates their understanding of the structure of the LAA and of potential positions for interventional devices within the LAA.

Some historic methods of realizing this desire include deriving an anatomical model of the LAA from imaging data, such as ultrasound data. The anatomical model can be processed to model the placement of closure devices with respect to the model, and an image of the anatomical model can be output with a representation of the modelled placement of the closure device. An example of such a process is described in US Patent Application No. US 2017/15719388, titled Left Atrial Appendage Closure Guidance in Medical Imaging.

There is an ongoing desire to improve a clinician's understanding of the structure of the LAA and of the potential position(s) for interventional devices within the LAA.

### SUMMARY OF THE INVENTION

The invention is defined by the claims.

According to examples in accordance with an aspect of the invention, there is provided a computer-implemented method of generating and displaying a rendering of a left atrial appendage of a patient.

The computer-implemented method comprises: obtaining, from an image processing system or memory, image data comprising a three-dimensional image of the left atrial appendage of the patient; obtaining, from an image processing system or memory, model data comprising an anatomical model of the left atrial appendage of the patient; determining, using the anatomical model, a potential position in the left atrial appendage of the patient for deriving one or more characteristics of an interventional device placable in the left atrial appendage of the patient; generating a rendering of the left atrial appendage of the patient using the image data; and displaying, at a user interface, the rendering of the left atrial appendage of the patient, wherein one or more visual parameters of the displayed rendering are based upon the determined potential position in the left atrial appendage of the patient.

The present disclosure proposes to adapt how a rendering of a left atrial appendage (LAA) is displayed based upon a determined potential position for deriving one or more characteristics of an interventional device within the LAA. Thus, rather than overlaying a visual representation of an interventional device (or other indicator usable for deriving characteristics of the interventional device) over a rendering, the rendering of the LAA itself can be modified or adapted based on the determined potential location usable for establishing one or more characteristics for the interventional device.

The present disclosure recognizes that a display of a rendering of an LAA can obscure or mask a visual representation of an interventional device, or indicator for deriving characteristics thereof, within the LAA or vice versa. By modifying a visual parameter of the rendered LAA based on the potential position, the likelihood that the rendering will obscure the visual representation of the interventional device is significantly reduced. This improves a human-machine interaction, and aids a user in their understanding of a potential location for deriving one or more characteristics for an interventional device within an LAA (in particular, by improving the availability of information that describes a relationship between a potential position usable to derive characteristics of an interventional device and the LAA).

The computer-implemented method therefore provides a visual aid for a clinician to aid their placement of an interventional device within the LAA.

As previously explained, the potential position is usable to derive one or more characteristics of the interventional device.

In some examples, the potential position is a potential position of the interventional device within the LAA, i.e. a position at which the interventional device would be located if placed in the LAA to perform its medical function. Thus, the one or more characteristics may comprise a position of the interventional device within the LAA.

In other examples, the potential position does not directly represent a position at which the interventional device may lie within the LAA (the “final position” of the interventional device), but rather is a position usable to derive characteristics of an interventional device to be placed in the LAA, such as a position of the interventional device, a type of the interventional device or a dimension of the interventional device. This can be referred to as a “reference position”.

For example, the potential position (reference position) may be a position in the LAA that is offset from (e.g. downstream or more distal than) the desired position for the interventional device. This can facilitate selection of an interventional device according to clinical or manufacturer guidelines, which could, for example, specify that the properties of the interventional device are to be selected based on a reference position in the LAA. For example, the reference position may be offset from the “final” position of the interventional device, e.g. to ensure a snug fit for the interventional device.

In either event, the potential position can be considered a potential position for the interventional device.

The anatomical model is preferably a three-dimensional model of at least the left atrial appendage of the patient (and may additionally include other structures of the heart). Methods of generating a model of a left atrial appendage of the patient will be well known to the skilled person, and may employ any suitable imaging technology (e.g. ultrasound, MRI or CT scanning) to generate image data that can be segmented to generate model data of the left atrial appendage.

Similarly, the image data comprises image data of the left atrial appendage, but may also include image data of other structures of the heart and surrounding tissue/structures. The one or more visual properties may comprise a rotation of the displayed rendering. The one or more visual properties may comprise a zoom level of the displayed rendering. The one or more visual properties may comprise a position and/or orientation of one or more cutting planes of the rendering.

For instance, the one or more visual properties may include a bounding box that identifies the volume of the patient's anatomy that is rendered for display.

These approaches can facilitate adjustment of the displayed rendering to reduce the presence of elements of the displayed rendering between the potential position and the viewing plane of the user interface (i.e. so that a user can see the potential position within the rendering of the LAA), or to other improve a contextual understanding around the potential position.

A cutting plane, sometimes called a cut plane or a section plane, is a plane that defines which elements of a model are made visible or rendered. For example, elements of the LAA on one side of a cutting plane may be made visible or rendered, whereas elements on the other side of the cutting plane may be made invisible or may not be rendered.

The step of determining a potential position for the interventional device optionally comprises receiving a user input signal indicating a user-desired position for the interventional device. The user input signal may be received by a user interface that displays the rendering of the left atrial appendage.

The one or more visual parameters may comprise a property of one or more pixels of the rendering that represent an area in the vicinity of the potential position, preferably wherein the property of the one or more pixels is a color property of the one or more pixels.

In particular, rather than overlaying a visual representation of an interventional device or a potential position for an interventional device over the rendering of the LAA, properties of pixels of the rendering itself can be modified. Put another way, rather than displaying a dedicated element for visually representing the interventional device, the rendering of the LAA may itself be modified to visual represent a potential location for the interventional device.

In particular embodiments, only pixels of the rendering that directly represent a part of the LAA that is in the vicinity of the potential position for the interventional device (e.g. a part of the LAA that would make contact with an interventional device positioned at that position) are modified. Thus, pixels of the rendering that do not represent the LAA in the vicinity of the potential position remain unmodified.

In some embodiments, the one or more pixels comprise only pixels representing tissue within the immediate vicinity of the potential position in the left atrial appendage. For example, the one or more pixels may comprise only pixels that represent tissue lying within a predetermined distance of the potential position (e.g. within 2 mm of the potential position or within 10 mm of the potential position). Other suitable predetermined distances would be apparent to the skilled person.

This approach reduces the obstructive effect that a representation of the interventional device provided by the user interface may have on the overall display. In particular, rather than overlaying the representation of the interventional device, the appearance of the LAA can be modified to illustrate the potential position for the interventional device without obstructing areas of the LAA to which the interventional device would not directly interact with (if positioned at the potential position).

In some embodiments, where the potential position represents a potential position of the interventional device, the one or more pixels may comprise only pixels representing tissue that would make contact with the interventional device positioned at the potential position and/or (optionally) tissue within the immediate vicinity thereof.

It is emphasized that it is not essential that all pixels representing tissue that would make contact with the interventional device (or in the immediate vicinity thereof) are modified as a result of the potential position. Rather, only some or a portion of these pixels may be modified.

In some embodiments, where the potential position represents a reference position of the interventional device, the one or more pixels may comprise only pixels representing tissue that would make contact with the reference position and/or (optionally) tissue within the immediate vicinity thereof.

For the avoidance of doubt, it is assumed that noise is negligible, and that the identification of pixels affected by the potential position excludes pixels that are affected due to noise.

The method may further comprise a step of processing the anatomical model to predict a model-derived size of an interventional device placable within the left atrial appendage of the patient, wherein one or more visual parameters of the displayed rendering are further based upon the predicted, model-derived size of the interventional device.

In some examples, the method further comprises predicting a rendering-derived size and/or shape of an interventional device placable in the left atrial appendage by processing the rendering of the left atrial appendage and the determined potential position.

Thus, rendering information can be used to perform an improved estimation of suitable size and/or shape characteristics for an interventional device to be placed in the LAA. A rendering may contain additional information (e.g. more granular or more specific information) about the potential position (compared to the model data alone), which can be used to improve an estimated size and/or shape for an interventional device to be positioned at the potential position.

The step of predicting a rendering-derived size and/or shape of the interventional device may comprise using pixel information of the rendering of the left atrial appendage to predict a size and/or shape of the interventional device.

The model data preferably comprises mesh data that represents an anatomical model of the left atrial appendage of the patient. The model data may be generated using a model-based segmentation approach.

Preferably, the interventional device comprises an occlusion device, i.e. an occluder, for the left atrial appendage.

There is also proposed a computer-implemented method of generating and displaying a rendering of a left atrial appendage of a patient, the computer-implemented method comprising: obtaining, from an imaging system, image data of the left atrial appendage of the patient; performing, using an image processing system, a segmentation process on the image data to generate model data comprising a model of the left atrial appendage of the patient; and performing the method previously described.

The step of obtaining image data may be identical to the previously described step of obtaining image data or be a completely separate step.

There is also proposed a computer program product comprising computer program code means which, when executed on a computing device having a processing system, cause the processing system to perform all of the steps of any herein described method.

There is also proposed a rendering system configured to generate and display a rendering of a left atrial appendage of a patient.

The rendering system comprises: a processor circuit configured to: obtain, from an image processing system or memory, image data comprising a three-dimensional image of the left atrial appendage of the patient; obtain, from an image processing system or memory, model data comprising an anatomical model of the left atrial appendage of the patient; determine, using the anatomical model, a potential position in the left atrial appendage of the patient for deriving one or more characteristics of an interventional device placable in the left atrial appendage of the patient; and generate a rendering of the left atrial appendage of the patient using the image data.

The rendering system also comprises a user interface configured to display the rendering of the left atrial appendage of the patient.

One or more visual parameters of the displayed rendering are based upon the determined potential position in the left atrial appendage of the patient.

In some examples, the processor circuit generates display data comprising the rendering for display by the user interface.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a flowchart illustrating a method according to an embodiment;

FIG. 2 illustrates a rendering generated by a method according to an embodiment;

FIG. 3 illustrates an anatomical model of a left atrial appendage for use in an embodiment;

FIG. 4 illustrates a method according to an embodiment;

FIG. 5 illustrates a rendering generated by a method according to an embodiment; and

FIG. 6 illustrates a rendering system according to an embodiment.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The present disclosure proposes a mechanism for generating a rendering of a left atrial appendage of a patient. The potential position usable for determining one or more characteristics of one or more interventional devices to be placed within the left atrial appendage is determined from model data that contains an anatomical model of the left atrial appendage. A rendering of the left atrial appendage is generated, from image data, and subsequently displayed. Visual parameters of the displayed rendering of the left atrial appendage are responsive to the determined potential position(s).

For the purposes of the present disclosure, the method and apparatus have been described in the context of renderings of left atrial appendages. The present invention is particularly advantageous when used for positioning interventional devices within a left atrial appendage, due to the geometry of the left atrial appendage introducing difficulty in planning the placement and selection (e.g. of size and shape) of an interventional devices.

Examples of suitable interventional devices include an occluder or closure device. Other suitable devices for placement in an anatomical structure would be apparent to the skilled person, such as a device with a similar delivery approach to a stent.

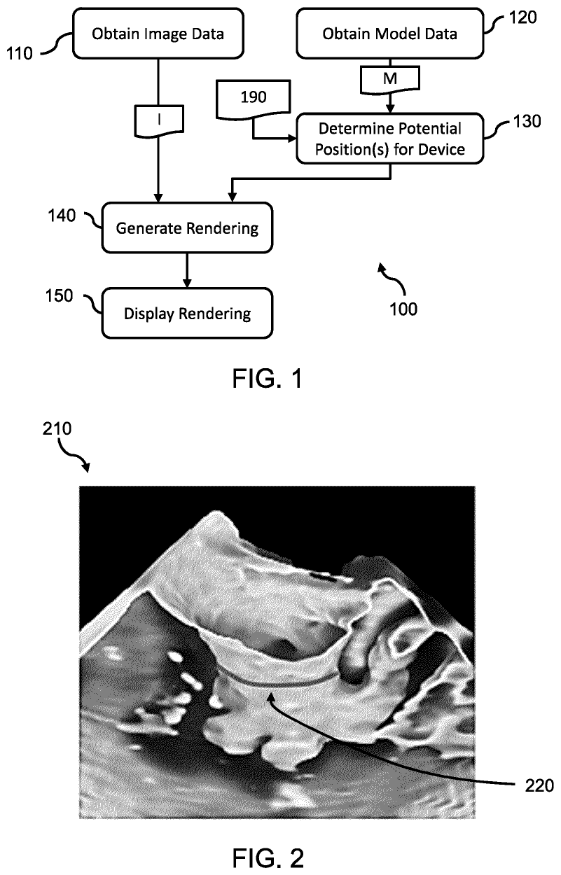

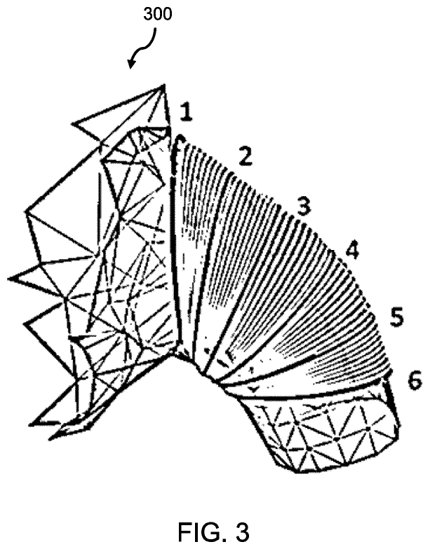

FIG. 1 is a flowchart illustrating a method 100 according to an embodiment. The method is configured for generating a rendering of a left atrial appendage (LAA). An example of a rendering of an LAA is illustrated in FIG. 2.

The method 100 is configured to generate and display a rendering of the LAA in which one or more visual parameters of the displayed rendering are based upon (i.e. responsive to) one or more potential positions usable to derive one or more characteristics for an interventional device placable in the LAA. Thus, the appearance of the rendering itself is dependent upon one or more potential positions with respect to the LAA.

The method 100 comprises a step 110 of obtaining image data (I). The image data comprises a three-dimensional image of the left atrial appendage of the patient. In other words, the image data comprises information that (if appropriately processed) facilitates display of a 3D rendering of a left atrial appendage of the patient.

Suitable image data would be readily apparent to the skilled person and may, for example, take the form of ultrasound data, MM data, CT scan data and so on. Any suitable data obtained from 3D imaging (at least) a left atrial appendage of the patient can be used as image data. The image data may, for instance, comprise a plurality of 2D images that form an overall 3D image, or may comprise a cloud of points that represent a 3D image of at least the LAA.

The three-dimensional image of the image data may also include other features or anatomical structures of the patient, e.g. surrounding heart tissue, surrounding bone/muscle structures and the like. In some examples, the three-dimensional image is an image of the entire chest of the patient, or of the entire body of the patient (e.g. a full-body 3D scan). Thus, the image data comprises a three-dimension image of at least the left atrial appendage of the patient.

In step 110, the image data may be obtained from any suitable image processing system or memory. For example, image data may be obtained from an ultrasound image processor, a CT image processor, a memory or storage facility and so on.

The method 100 further comprises a step 120 of obtaining model data (M). The model data comprises an anatomical model of the left atrial appendage of the patient. The model data may, for example, represent a segmentation result of an image segmentation algorithm performed on some image data of the patient, e.g. performed on the image data obtained in step 110.

By way of example, the model data may comprise mesh data that represents an anatomical model of the left atrial appendage of the patient. A mesh may have a defined number of vertices and triangles. Further anatomical information may be attached to specific regions. For example, each triangle may carry information that it belongs to a certain part of the mesh.

It is known to segment an anatomical structure in a medical image using a deformable model that is adapted to the specific image data. This form of segmentation is also referred to as model-based segmentation. The deformable model may define a geometry of a generic or average anatomical structure, e.g., in the form of a multi-compartmental mesh of triangles. Inter-patient and inter-phase shape variability may be modeled by assigning an affine transformation to each part of the deformable model.

One approach for performing segmentation on image data (namely CT image data) is disclosed by Ecabert, O.; Peters, J.; Schramm, H.; Lorenz, C.; von Berg, J.; Walker, M.; Vembar, M.; Olszewski, M.; Subramanyan, K.; Lavi, G. & Weese, J. Automatic Model-Based Segmentation of the Heart in CT Images Medical Imaging, IEEE Transactions on, 2008, 27, pp. 1189-1201.

Other segmentation approaches use deep learning, e.g. machine-learning processes such as neural networks, in order to perform segmentation on an image. One example is shown by Ronneberger, Olaf, Philipp Fischer, and Thomas Brox. “U-Net: Convolutional Networks for Biomedical Image Segmentation.” ArXiv:1505.04597 [Cs], May 18, 2015. Another example is shown by Çiçek, Özgün, Ahmed Abdulkadir, Soeren S. Lienkamp, Thomas Brox, and Olaf Ronneberger. “3D U-Net: Learning Dense Volumetric Segmentation from Sparse Annotation.” In Medical Image Computing and Computer-Assisted Intervention—MICCAI 2016, edited by Sebastien Ourselin, Leo Joskowicz, Mert R. Sabuncu, Gozde Unal, and William Wells, 424-32. Lecture Notes in Computer Science. Springer International Publishing, 2016.

The model data may be obtained from an image processing system (e.g. that processes image data using a segmentation algorithm) and/or a memory.

The spatial relationship between the model data and the 3D image of the LAA may be predetermined or calculable. Methods of determining a spatial relationship between model data and a 3D image are well known to the skilled person (e.g. by identifying landmarks or the like). Of course, if model data is generated directly from the image data (e.g. via segmentation), then the spatial relationship is already known.

Landmark detection could, for example, use a deep learning landmark detection approach such as that disclosed by Zhang, Zhanpeng, Ping Luo, Chen Change Loy, and Xiaoou Tang. “Facial Landmark Detection by Deep Multi-Task Learning.” In Computer Vision—ECCV 2014, edited by David Fleet, Tomas Pajdla, Bernt Schiele, and Tinne Tuytelaars, 94-108. Lecture Notes in Computer Science. Cham: Springer International Publishing, 2014.

The method 100 also comprises a step 130 of determining, using the anatomical model, a potential position in the left atrial appendage of the patient for deriving one or more characteristics of an interventional device placable within the left atrial appendage. In other words, the model data is processed in order to identify one or more potential positions within the left atrial appendage.

A potential position may be a position in which an appropriate interventional device (if placed at the position) is able to carry out its intended medical function, e.g. with a minimum efficiency requirement.

In other examples, a potential position may be a position that can be used to derive or calculate one or more characteristic of an interventional device that, when placed in the LAA, can carry out its intended medical function. Thus, the potential position may be a “reference position”, usable for calculating characteristics of the interventional device which is not to be positioned at the potential position.

For example, the potential position may be a position offset from a position at which an interventional device can carry out its intended medical function, but can be used to derive suitable measurements or other characteristics for the interventional device.

The one or more characteristics may include, for example, a potential position for the interventional device; a size and/or shape of the interventional device and/or a type of the interventional device.

Preferably, a potential position comprises data that defines a plane of the LAA usable for deriving one or more characteristics for the interventional device. For example, the potential position may define a plane in which an interventional device can be positioned with respect to the LAA where it is able to carry out its intended medical function, or a position that defines characteristics of an interventional device to be placed in the LAA.

In some examples, the potential position comprises data that identifies a surface or areas of the LAA (e.g. as represented by the model data) that can be used to derive one or more characteristics for the interventional device. This may be a surface with which the interventional device makes contact if positioned in the LAA or a surface usable to derive characteristics of an interventional device not positioned on the surface.

Preferably, the potential position may represent a surface or areas with which an interventional device makes contact and/or is mounted when able to carry out its medical function. This surface, or these areas, can be labelled a “potential landing zone” for the interventional device, as they define the areas of the LAA with which the interventional device makes contact.

It is possible to derive a plane in which an interventional device can be positioned from a potential landing zone for an interventional device and vice versa.

For example, in a scenario in which the anatomical model is a mesh of triangles, a regression plane could be fitted to all triangles that are identified to belong to a certain potential landing zone for an interventional device. In particular, a regression plane could be fitted to the points (“pi,seg”) of all triangles if they belong to a part of a surface of an anatomical model of the LAA that is identified as a potential landing zone.

Similarly, if a plane for an interventional device is known, then the areas of the LAA that intersect the plane (e.g. in the model data or the image data) represent a “potential landing zone” for the interventional device.

In some examples, different potential positions may be encoded into the model data. In particular examples, pre-encoded landing zones, being areas of the LAA to which the interventional device makes contact, for interventional devices may be included in an anatomical model/mesh which is fitted to an LAA of a patient using the image data.

For example, during a segmentation algorithm, a mean model/mesh (which is to be adapted to the specific patient) may include/identify potential positions for an interventional device. The adaptation of the model may result in the pre-encoded potential positions being adapted for the specific patient, thereby facilitating simple determination of the potential position of an interventional device with respect to the LAA.

In some examples, additional potential positions in-between the pre-encoded (and adapted) potential positions may be derived by interpolating the pre-encoded potential positions using the model data.

This approach is illustrated in FIG. 3, which conceptually depicts an anatomical model 300 of the LAA (formed in the model data). This anatomical model has been adapted to fit image data of a patient, i.e. has been made patient specific. Pre-encoded positions for the potential position(s) are indicated using reference numerals 1 to 6. Interpolated positions are also illustrated. The locations/sizes of the interpolated positions are derived from the model data to follow the structure of the anatomical model of the LAA.

Turning back to FIG. 2, as another example, the step 130 of determining a potential position may comprise, for example, obtaining information about an interventional device or devices (e.g. a size and/or shape of possible interventional devices) and processing the model data to identify a position at which each interventional device can perform its/their medical function(s).

In some examples, a potential position for an interventional device may be established by modelling different possible interventional devices within the anatomical model of the LAA to automatically determine a possible position for an interventional device.

As an example, an interventional device may be represented by an ellipse of a certain size and/or shape. The model data may be processed to identify a position in the anatomical model of the LAA in which the ellipse would make contact with the surface of the anatomical model of the LAA. The position of the ellipse can thereby define the potential position.

In some examples, a user input 190, e.g. carried by a user input signal, is used to define one or more potential positions for the interventional device in step 130. This user input may be carried by a user input signal, and may indicate a user-desired position for the interventional device or a user-desired reference position.

Thus, step 130 may comprise receiving a user input signal indicating a user-desired position for the interventional device.

For example, a user may be able to select one or more of a plurality of automatically identified potential positions to act as the determined potential position(s). By way of example, a user input may define one or more desired relative positions within the left atrial appendage (e.g. whether a more proximal or more distal position with respect to the ostium is desired). Thus, a user may influence the (location of the) potential position.

In other words, the “potential position” may be a “user-selected potential position” from a variety of automatically identified potential positions. The user input may, for example, be received from any suitable user input interface.

By way of example, a user interface may provide one or more user interface elements (such as a slider) which can be controlled by a user to identify a relative position (e.g. more distal or more proximal with respect to the ostium) in the LAA. The method may be configured to respond to this indication by determining a new potential position or selecting an automatically identified potential position as the determined potential position (for further processing).

As another example, the user may be able to define characteristics of the interventional device, such as a desired orientation (e.g. with respect to other features of the heart), size and/or shape of the interventional device. From this information, the method may be configured to automatically select an appropriate position for the interventional device having the desired characteristics, e.g. a position in which the interventional device can carry out its medical function. This automatic selection may be performed be selecting a previously automatically determined potential position or by modelling the interventional device in the anatomical model, to generate a new potential position.

By way of example, a user interface may provide one or more user interface elements (such as a slider) which can be controlled by a user to define one or more characteristics of the interventional device, such as a relative angle of the interventional device.

Turning back to FIG. 1, the method 100 also comprises a step 140 of generating a rendering of the LAA of the patient using the image data.

A process of rendering is typically a process that converts 3D image data into a 2D image for display by a display or user interface. This may be a known volume rendering process. The output of a step of rendering is 2D image data, i.e. a 2D projection with respect to a camera, of the LAA for display by the display or user interface. The skilled person would be capable of using any suitable rendering approach for generating a rendering of the LAA.

The rendering may comprise, for example, 2D image data that defines values for pixels for display by a display or user interface, e.g. defining a color, opacity, intensity, saturation, hue, shading and/or other suitable pixel values.

In preferable examples, the rendering is generated in a way that the tissue/blood interface surface of the LAA can be visualized separately from the pericardium. In other words, all tissue (and fluid) around the LAA may be made transparent. This can be facilitated by using the model data to identify portions of the image data that represent areas of the interface surface of the LAA. This approach can improve a clinician's understanding of the LAA, by avoiding the inclusion of potentially obstructive features in the rendering.

In some example, the model data is processed to identify areas of the LAA (and surrounding tissue) to be rendered. This may be performed by establishing a bounding box that defines the tissue to be rendered as a rendering. The bounds of the bounding box may, at least in part, be defined by the potential position (as set out below).

The method 100 also comprises a step 150 of displaying the generated rendering at a user interface. Methods of displaying renderings are well known to the skilled person.

In some examples, the step of generating a rendering in step 140 comprises generating display data for the user interface to display. The display data may, for example, define properties for different pixels of a display of the user interface. Suitable methods would be readily apparent to the skilled person.

The method 100 is adapted such that one or more visual parameters of the displayed rendering are based upon the determined potential position(s) within the left atrial appendage of the patient.

In particular examples, the rendering is adjusted such that (visual) interference between itself and any displayed information from the anatomical context of the interventional device is reduced (e.g. by adjusting rotation, zoom and/or cutting planes of the rendering).

By modifying the visual parameters of the displayed rendering based on the determined potential position(s), the viewer of an image is provided with additional information that allows them to more accurately or more intuitively understand the potential position.

This provides an improved display of the LAA, to provide a visual aid for a clinician. For example, where the potential position represents a potential position for the interventional device within the LAA, this can credibly assist a clinician in the placement of the interventional device, by improving the clinician's understanding of potential positions for the interventional device within the LAA, allowing for more precise selection and positioning of an interventional device.

In particular, the step 140 is adapted so that the rendering of the LAA is further based upon one or more determined potential positions. In other words, the appearance of the LAA provided by the rendering of the LAA is modified based upon the determined potential position(s) within the left atrial appendage.

The present disclosure contemplates various possible visual parameters that could be modified based upon the determined potential position(s).

Optionally, the one or more visual properties comprises a rotation of the displayed rendering. In other words, the viewing angle of the LAA, i.e. the angle at which the LAA is rendered, may be dependent upon the determined potential position(s).

By way of example, the camera position and orientation of the rendering could be adjusted such the displayed rendering appears to look at a predetermined angle with respect to a plane defining the potential position, e.g. normal to the potential position or at a 45° angle to the normal. The plane defining the potential position may, for example, be a plane in which the interventional device lies if positioned at the potential position.

Optionally, the one or more visual properties comprises a zoom level of the displayed rendering. Thus, the relative size of the LAA in the rendering may be adjusted.

By way of example, if a potential position is further away (in 3D space) from the virtual camera of the rendering, the zoom level of the rendering may be increased and vice versa.

Optionally, the one or more visual properties comprises a position and/or orientation of a cutting plane of the rendering. In other words, one or more elements of the patient's anatomy can be excluded from the rendering based upon the potential position(s).

This approach can automatically reduce visual obstruction between the camera (of the rendering) and the potential position. This improves a clinician's view of the potential position, and thereby credibly aids the clinician's understanding of the potential position.

By way of example, tissue that is proximal (or distal, e.g. depending upon the position of the camera for the rendering) to the potential position could be excluded from the rendering. This way, the displayed position is less obstructed by rendered tissue.

The cutting plane could be configured to not be directly at the potential position, but rather at some offset d to improve a contextual understanding of the location of the potential position, whilst reducing obstruction.

As another example, the one or more visual parameters may comprise a bounding box that defines the tissue that is rendered in the rendering. The bounds of the bounding box may therefore be defined, at least partly, by the potential position. For example, a bounding box may be defined by positioned a predetermined bounding box volume with respect to the potential position (e.g. forming a center of the bounding box or other predetermined position within the bounding box).

A bounding box is effectively defined as a plurality of cutting planes of the rendering that define tissue that is excluded from the rendering.

Optionally, the one or more visual parameters comprise a property of one or more pixels of the rendering that represent an area in the vicinity of the potential position, preferably wherein the property of the one or more pixels is a color property of the one or more pixels.

In other words, one or more property of pixels of the rendering (proximate to the interventional device) may be modified based upon the potential position. In particular examples, a color or opacity of a pixel of the rendering may be modified.

In particular, values for pixels that directly represent tissue of the patient in the vicinity may be modified. For example, values for pixels that directly represent tissue in the immediate vicinity of the potential position may be modified.

In particular examples, only pixels of the rendering that directly represent tissue that is in the vicinity of the potential position(s) (e.g. a part of the LAA that would make contact with an interventional device positioned at that position) may be modified. In some examples, only values for pixels that represent parts tissue that form or are in the vicinity of a potential landing zone for the interventional device may be modified.

For example, the method may be configured to only modify pixel properties for pixels that represent tissue that is closer to the potential position (e.g. plane or landing zone) than some minimal threshold (e.g. 2 mm).

As an example, the method may be configured to only modify pixel properties for pixels that represent tissue to which the interventional device would make contact if positioned at the potential position (i.e. the “potential landing zone” of the interventional device).

Thus, rather than superimposing an additional rendering of an interventional device (or potential position) over a rendering of the LAA, properties of the rendering itself are altered to provide additional information about the position of the interventional device.

Simply superimposing an additional rendering would result in other pixels, which do not represent tissue in the vicinity of the potential position, having modified properties. The present disclosure proposes to only modify properties for pixels which directly represent tissue in the vicinity of the potential position.

Thus, only a single rendering is generated, rather than multiple renderings which overlay one another.

From the foregoing, it will be apparent that the potential position(s) may affect the rendering of the LAA from the 3D image data.

Of course, the rendering may be updated if the potential position(s) are modified, e.g. a new potential position is selected by a user (which could lead to an automatic modification to the size of the interventional device) or the user changes the characteristics of the interventional device (which could lead to an automatic update of the potential position).

FIG. 2 illustrates a display of a rendering 210 of an LAA provided by an embodiment of the invention. One or more visual parameters of the rendering 210 have been modified based upon the potential position. In the illustrated example, the potential position is a potential position for the interventional device within the LAA, i.e. a position at which the interventional device would be placed to perform its medical function.

In particular, a color property of pixels of the rendering 210 have been modified in the vicinity of the potential position in order to draw attention to the potential position with respect to the LAA without obstructing other parts of the rendering.

The modified pixels are visible in a modified section 220 of the rendering 210.

In particular, it will be seen that pixel properties are only modified for pixels that represent a part of the LAA in the vicinity of the potential position for the interventional device e.g. representing part of a potential landing zone for an interventional device. This differs from simply overlapping a representation of the interventional device over the rendering (e.g. overlaying an ellipse representing an interventional device), which could modify pixels that do not represent a part of the LAA in the vicinity of the potential position(s).

Moreover, a viewing angle and cutting plane of the rendering 210 have been selected based upon the potential position.

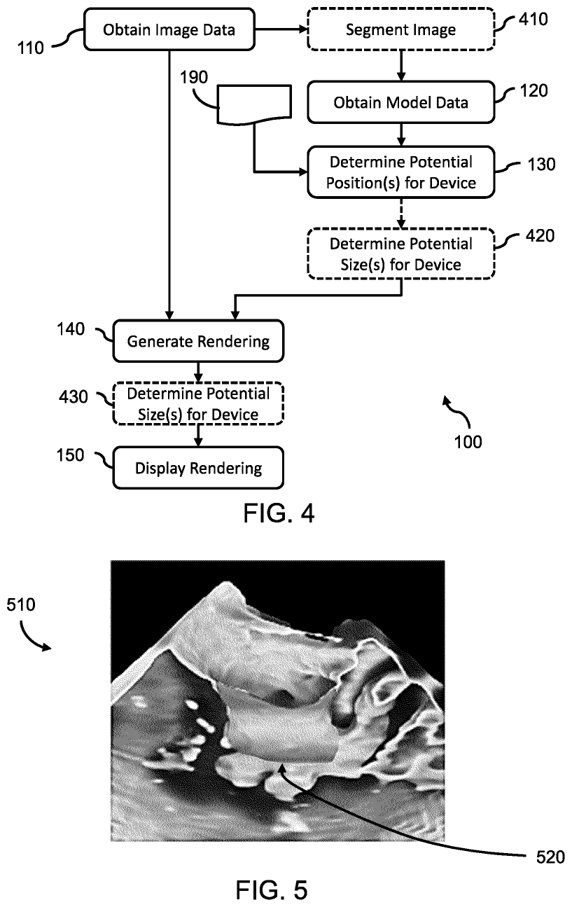

FIG. 4 illustrates a method 400 in which additional (optional) steps are included.

In particular, the method 400 further comprises an optional step 410 of segmenting the image data in order to obtain the model data. Processes for segmenting image data have been previously described, and can be employed for carrying out step 410. If step 410 is omitted, the model data can be obtained from an external image processing system or memory.

For example, a model-based segmentation may be used to generate the model data. In some examples, a mean model or mesh of the LAA and surrounding regions (e.g. of the complete heart including the LAA) is adapted to the patient's image to produce a personalized version of the mode/mesh.

The mean model/mesh may include, for example, potential positions for an interventional device. The adaptation of the model may result in the potential positions being adapted for the specific patient, thereby facilitating simple determination of the potential position with respect to the LAA.

The method 300 further comprises an optional step 420 of determining a size for the interventional device from the model data (obtained in step 120). This can be carried out at the same time as identifying a potential position for the interventional device.

Thus, optional step 420 comprises processing the anatomical model (or model data) to predict a model-derived size of an interventional device placable in the left atrial appendage using a potential position within the left atrial appendage of the patient. An example of step 420 will be provided in the context of scenario in which the potential position directly represents a potential position for the interventional device in the LAA, i.e. a position at which the interventional device will be located if placed in the LAA and able to perform its medical function.

In this scenario, step 420 can be performed by conceptually fitting a model of the interventional device into the anatomical model of the LAA at the potential position for the interventional device, and measuring dimensions of or performing a geometric analysis on the model of the interventional device. In some examples, the model is an ellipse. In other examples, the model is a mesh representing an interventional device.

By way of example, the interventional device may be represented by an ellipse. The model data can then be processed to identify the maximum size and/or shape of an ellipse that can be placed at a potential position within the LAA (whilst fitting in the LAA). This may be performed by conceptually fitting an ellipse into the anatomical model at the potential position, and measuring dimensions of the ellipse. The dimensions may include, for example, a size of a major and minor axis for the fitted ellipse. The fitted ellipse may (also) define a coordinate system with the center point and three directions (major axis, minor axis, normal).

If the anatomical model is a triangular mesh that represents the LAA of the patient, then a size of the interventional device can be predicted by identifying (points “pi,seg” of) the triangles that represent a potential landing zone for the interventional device, e.g. using a plane or based on pre-encoded positional data, and measuring dimensions of a model of an interventional device (e.g. an ellipse) that makes contact with these triangles or the points thereof.

Thus, anatomical measurements of the LAA at the potential position could be determined, and thereby used as a basis for identifying the size of the interventional device. For example, the anatomical measurement could be presented to a clinician to aid the clinician's decision on the size for the interventional device.

Another example of step 420 will be provide in the context of a scenario in which the potential position represents a reference position used for deriving characteristics of an interventional device (but does not directly represent a position at which the interventional device is to be placed).

In this scenario, a similar approach may be made for calculating dimensions of the LAA at the potential position. The dimensions of the LAA may then be usable to derive characteristics of the interventional device to be placed at the potential position. Purely by way of example, in order to determine the appropriate size for some interventional devices, it may be required to measure the dimensions of the LAA at a predetermined distance from the desired position for the interventional device (e.g. 10 mm more proximal or 20 mm more distal) or over a wider range of distances. This may be set out in manufacturer's guidance. One or more visual parameters of the displayed rendering may be further based upon the predicted, model-derived size of the interventional device.

Thus, the displayed rendering may further carry information about the size of an interventional device that can be placed in the LAA, e.g. at the potential position or positions. For example, different colors of the pixels of the rendering may indicate different sizes for an interventional device. As an example, the greater the dimensions of the interventional device, the more red (and less green) an interventional device appears, and vice versa.

In some examples, step 420 may further comprise presenting information responsive to an identified size and/or shape of the interventional device may be presented to a user at a user interface. The information may, for example, comprise information on a predicted size for the anatomical device. This provides a clinician with useful clinical information for selection of an appropriate interventional device that can be placed at the potential position.

In some examples, the method further comprises a step 430 of predicting a size and/or shape of an interventional device placable in the LAA using the rendering of the left atrial appendage and the determined potential position. This predicted size and/or shape can be a “rendering-derived” size or shape.

Thus, rendering information can be used to perform an improved estimation of suitable size and/or shape characteristics for an interventional device to be placed at the LAA. A rendering may contain additional information (e.g. more granular or more accurate) information about the potential position (compared to the model data alone), which can be used to improve an estimated size and/or shape for an interventional device.

The rendering information may be used, for example, to refine a model-derived size and/or shape of the interventional device generated in step 420.

By way of example, to refine the measurements obtained from the anatomical model, it is possible to use selected pixels from the volume rendering. It is possible to use information of pixels that are proximate to the potential position, e.g. have undergone a change of property (e.g. due to the determined potential position), to improve an identification of the size/shape of the interventional device.

In particular, the positions of pixels “pi,vox” (which are proximate to the potential position, e.g. have been modified as a result of the potential position) can be used to refine the geometrical analysis that was previously performed on points “pi,seg” derived from the anatomical model alone. The positions “pi,vox” can be either be used alone for a new sizing (e.g. ellipse fitting) or in combination to the segmented points “pi,seg”. The initial ellipse could stabilize the selection process of points “pi,vox”, e.g. to only accept points not further from the initial ellipse than a threshold r. Further, a weighting factor w could determine the weighting between the segmented points and the pixel points.

For the sake of completeness, the inventors also envisage that the concept of using rendering information to refine a model-derived size and/or shape of the interventional device may, by itself, be a stand-alone invention. Thus, there may be proposed a concept of obtaining a rendering of a LAA, the rendering including information on a potential position for an interventional device, and processing the rendering (optionally with model data) to determine one or more characteristics of the interventional device.

In some examples, step 430 may further comprise presenting information responsive to an identified size and/or shape of the interventional device at the potential position may be presented to a user at a user interface.

The above description has been generally focused upon modifying a rendering based upon a single potential position for an interventional device, it will be apparent that a rendering could be modified based upon different potential positions (e.g. for different sized interventional devices).

As an example, the rendering of the LAA could be modified based upon the size of each of a plurality of interventional devices as derived from their respective potential positions. As an example, the method may comprise color-coding representative pixels for the LAA tissue according to the size of an interventional device that would make contact with the LAA tissue if positioned to perform its medical function, e.g. blue for smaller regions and red for wider regions.

This concept is illustrated by FIG. 5, which illustrates a display of a rendering 510 of an LAA provided by an embodiment of the invention. Visual parameters of the rendering 510 has been modified based upon the potential positions of a plurality of different interventional devices.

In particular, a color property of pixels of the rendering 510 have been modified in the vicinity of a potential position for each interventional device in order to draw attention to the potential position with respect to the LAA without obstructing other parts of the rendering.

The modified pixels are visible in a modified section 520 of the rendering 510. The modification to a color property is dependent upon the size of a most proximate interventional device positioned at its potential position. In the illustrated example, the darker a pixel, the larger the interventional device (positioned at its potential position) that is most proximate to the pixel of the LAA.

As before, it will be seen that pixel properties are only modified for pixels that represent a part of the LAA in the vicinity of the potential position for the interventional device, e.g. representing part of a potential landing zone for an interventional device. This differs from simply overlapping a representation of the interventional device over the rendering (e.g. overlaying an ellipse representing an interventional device), which could modify pixels that do not represent a part of the LAA in the vicinity of the potential position(s).

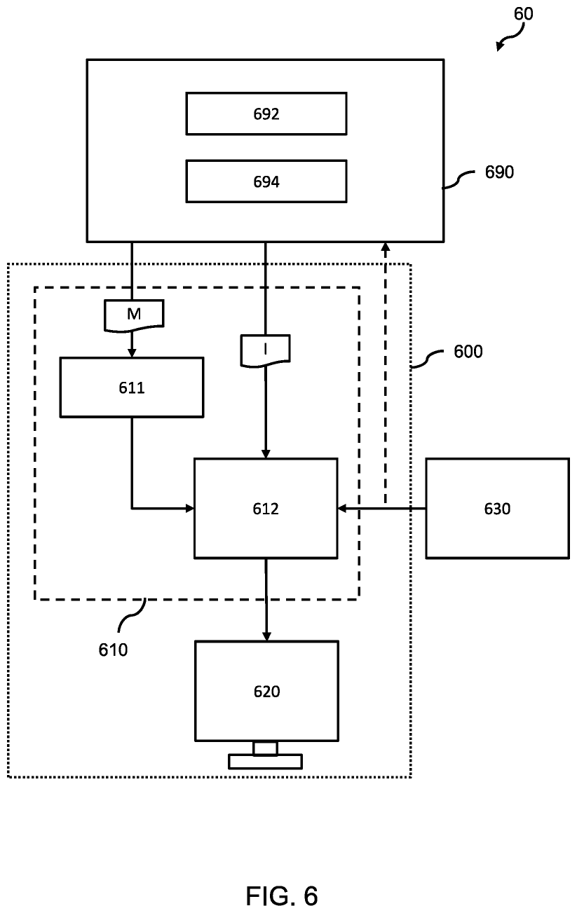

FIG. 6 illustrates a system 60 comprising a rendering system 600 according to an embodiment of the invention. The rendering system 600 is configured to generate and display a rendering of a left atrial appendage of a patient.

The rendering system comprises a processor circuit 610.

The processor circuit is configured to obtain, from an image processing system 690 or memory, image data I comprising a three-dimensional image of the left atrial appendage of the patient.

The processor circuit 610 is also configured to obtain, from the image processing system or memory, model data M comprising an anatomical model of the left atrial appendage of the patient.

The processor circuit 610 is also configured to determine, using the anatomical model, a potential position for an interventional device within the left atrial appendage of the patient.

The processor circuit 610 is also configured to generate a rendering of the left atrial appendage of the patient using the image data. One or more visual parameters of the (displayed) rendering are based upon the determined potential position for the interventional device within the left atrial appendage of the patient.

The processor circuit may comprise a model analysis unit 611 to perform the determining of the potential position for the interventional device using the anatomical model. Similarly, the processor circuit 610 may comprise an image renderer 612 configured to generate the rendering using the image data. However, these are only conceptual, and the skilled person would recognize that their tasks could be carried out by any suitable component or module.

The rendering system 600 also comprises a user interface 620 configured to display the rendering of the left atrial appendage of the patient. The user interface 620 may comprise a visual display (e.g. a screen) for displaying the rendering.

The rendering system 600 may be appropriately configured to carry out any method described in this document. The skilled person would be readily capable of adapting the rendering system 600 (and any units forming the rendering system 600) appropriately.

The system 60 may further comprise an image processing system 690. The image processing system 690 may be configured to generate the image data I (e.g. using information received from a patient scanner, such as an ultrasound machine) and/or generate the model data M, e.g. by performing a segmentation algorithm on the image data. The image processing system may comprise an image generator 692, for generating the image data I, and an image segmenter 694, for generating the model data M. The image segmenter may be configured to perform a segmentation algorithm on the image data.

The system 60 may further comprise a user input interface 630, for receiving a user input. This may allow the user to select a potential position for an interventional device, e.g. from a choice of automatically generated potential positions, or define characteristics of the interventional device for defining the potential position.

In some examples, the user input interface 630 may be integrated into the user interface 620.

Other uses for the user input interface 630 will be apparent to the skilled person, e.g. to override visual parameters of the rendering selected by the processor circuit 610.

The skilled person would be readily capable of developing a processing system for carrying out any herein described method. Thus, each step of the flow chart may represent a different action performed by a processing system, and may be performed by a respective module of the processing system.

Embodiments may therefore make use of a processing system. The processing system can be implemented in numerous ways, with software and/or hardware, to perform the various functions required. A processor or processor circuit is one example of a processing system which employs one or more microprocessors that may be programmed using software (e.g., microcode) to perform the required functions. A processing system may however be implemented with or without employing a processor, and also may be implemented as a combination of dedicated hardware to perform some functions and a processor (e.g., one or more programmed microprocessors and associated circuitry) to perform other functions.

Examples of processing system components that may be employed in various embodiments of the present disclosure include, but are not limited to, conventional microprocessors, application specific integrated circuits (ASICs), and field-programmable gate arrays (FPGAs).

In various implementations, a processor or processing system may be associated with one or more storage media such as volatile and non-volatile computer memory such as RAM, PROM, EPROM, and EEPROM. The storage media may be encoded with one or more programs that, when executed on one or more processors and/or processing systems, perform the required functions. Various storage media may be fixed within a processor or processing system or may be transportable, such that the one or more programs stored thereon can be loaded into a processor or processing system.

It will be understood that disclosed methods are preferably computer-implemented methods. As such, there is also proposed the concept of a computer program comprising code means for implementing any described method when said program is run on a processing system, such as a computer. Thus, different portions, lines or blocks of code of a computer program according to an embodiment may be executed by a processing system or computer to perform any herein described method.

In some alternative implementations, the functions noted in the block diagram(s) or flow chart(s) may occur out of the order noted in the figures. For example, two blocks shown in succession may, in fact, be executed substantially concurrently, or the blocks may sometimes be executed in the reverse order, depending upon the functionality involved.

In the context of the present disclosure, all images are medical images.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. If a computer program is discussed above, it may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted the term “adapted to” is intended to be equivalent to the term “configured to”. Any reference signs in the claims should not be construed as limiting the scope.

1. A computer-implemented method of generating and displaying a rendering of a left atrial appendage of a patient, the computer-implemented method comprising:
obtaining, from an image processing system or memory, image data (I) comprising a three-dimensional image of the left atrial appendage of the patient;
obtaining, from an image processing system or memory, model data (M) comprising an anatomical model of the left atrial appendage of the patient;
determining, using the anatomical model, a potential position in the left atrial appendage of the patient for deriving one or more characteristics of an interventional device placable in the left atrial appendage of the patient;
generating a rendering of the left atrial appendage of the patient using the image data; and
displaying, at a user interface, the rendering of the left atrial appendage of the patient,
wherein one or more visual parameters of the displayed rendering are based upon the determined potential position in the left atrial appendage of the patient.

2. The computer-implemented method of claim 1, wherein the potential position is a potential position for the interventional device within the left atrial appendage of the patient.

3. The computer-implemented method of claim 1, wherein the one or more visual properties comprises: a rotation of the displayed rendering; a zoom level of the displayed rendering and/or a position and/or orientation of a cutting plane of the rendering.

4. The computer-implemented method of claim 1, wherein the step of determining a potential position for the interventional device comprises receiving a user input signal indicating a user-desired position for the interventional device, preferably wherein the user input signal is received by a user interface that displays the rendering of the left atrial appendage.

5. The computer-implemented method of claim 1, wherein the one or more visual parameters comprise a property of one or more pixels of the rendering that represent an area in the vicinity of the potential position, preferably wherein the property of the one or more pixels is a color property of the one or more pixels.

6. The computer-implemented method of claim 5, wherein the one or more pixels comprise only pixels representing tissue within the immediate vicinity of the potential position in the left atrial appendage.

7. The computer-implemented method of claim 1, further comprising a step of processing the anatomical model to predict a model-derived size of an interventional device placable within the left atrial appendage of the patient,
wherein one or more visual parameters of the displayed rendering are further based upon the predicted, model-derived size of the interventional device.

8. The computer-implemented method of claim 1, further comprising predicting a rendering-derived size and/or shape of an interventional device placable in the left atrial appendage by processing the rendering of the left atrial appendage and the determined potential position.

9. The computer-implemented method of claim 8, wherein the step of predicting a rendering-derived size and/or shape of the interventional device comprises using pixel information of the rendering of the left atrial appendage to predict a size and/or shape of the interventional device.

10. The computer-implemented method of claim 1, wherein the model data comprises mesh data that represents an anatomical model of the left atrial appendage of the patient, preferably wherein the model data is generated using a model-based segmentation approach.

11. The computer-implemented method of claim 1, wherein the interventional device comprises an occlusion device for the left atrial appendage.

12. A computer-implemented method of generating and displaying a rendering of a left atrial appendage of a patient, the computer-implemented method comprising:
obtaining, from an imaging system, image data of the left atrial appendage of the patient;
performing, using an image processing system, a segmentation process on the image data to generate model data comprising a model of the left atrial appendage of the patient; and
performing the method of claim 1.

13. A computer program product comprising computer program code means which, when executed on a computing device having a processing system, cause the processing system to perform all of the steps of the method according to claim 1.

14. A rendering system configured to generate and display a rendering of a left atrial appendage of a patient, the rendering system comprising:
a processor circuit configured to:
obtain, from an image processing system or memory, image data (I) comprising a three-dimensional image of the left atrial appendage of the patient;
obtain, from an image processing system or memory, model data (M) comprising an anatomical model of the left atrial appendage of the patient;
determine, using the anatomical model, a potential position in the left atrial appendage of the patient for deriving one or more characteristics of an interventional device placable in the left atrial appendage of the patient;
generate a rendering of the left atrial appendage of the patient using the image data; and
a user interface configured to display the rendering of the left atrial appendage of the patient,
wherein one or more visual parameters of the displayed rendering are based upon the determined potential position in the left atrial appendage of the patient.

15. The rendering system of claim 14, wherein the processor circuit generates display data comprising the rendering for display by the user interface.
